(19) United States
(12) Patent Application Publication
(10) Pub. No.: US 2025/0160614 A1
(43) Pub. Date: May 22, 2025

(54) SYNCHRONIZED MOTION OF INDEPENDENT SURGICAL DEVICES TO MAINTAIN RELATIONAL FIELD OF VIEWS

(71) Applicant: Cilag GmbH International, Zug (CH)

(72) Inventors: Frederick E. Shelton, IV, Hillsboro, OH (US); Kevin M. Fiebig, Cincinnati, OH (US); Charles J. Scheib, Loveland, OH (US)

(21) Appl. No.: 18/810,170

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 1/00; A61B 1/313; G06T 7/20; G06T 7/30

(52) U.S. Cl. CPC A61B 1/00009; A61B 1/0005; G06T 7/20; G06T 7/30; A61B 1/3132; G06T 2207/10016; G06T 2207/10068; G06T 2207/10081; G06T 2207/30004; G06T 2207/30244

(57) ABSTRACT

Devices, systems, and techniques for synchronized motion of independent surgical devices to maintain relational field of views. A first imaging device may receive a video data stream captured by a second imaging device. The first imaging device may be located on a first side of an anatomical barrier and the second imaging device may be located on a second side of the anatomical barrier. The first side and the second side may be opposing sides of the anatomical barrier. The first imaging device may determine, based on the video data stream, a coupled field of view between the first imaging device and the second imaging device. The first imaging device may determine, based on the video data stream, that the second imaging device has moved. The first imaging device may adjust an imaging parameter of the first imaging device to maintain the coupled field of view.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/809,890, filed Aug. 20, 2024, and
U.S. patent application Ser. No. 18/810,133, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

In operating rooms, multiple surgical imaging devices may operate in close proximity to one another. In addition, the imaging devices may all be from different manufacturers and may have different control systems. The imaging devices may not be aware of the presence of other devices. Even if the devices are aware of other devices, the devices may not be able to communicate to coordinate their actions. This lack of coordination may limit the field of view of a user (e.g., surgeon). This limited visibility may cause the user to miss important events during surgery, such as an unintended bleed.

Synchronized imaging of two system may be used to maintain a common field-of-view or perspective for both systems. The synchronized imaging may allow a user to seamlessly transition objects from one field of view to another. Synchronized visualization may involve synchronized motion of the cameras. Synchronized visualization may involve electronic and/or algorithmic field-of-view limiting and/or overlapping imaging to enable each camera to capture a larger field of view than originally possible. The two systems may produce a composite image by adapting the synchronized imaging arrays.

Multiple scopes may use synchronized motion to maintain a relational field-of-view. For example, independent imaging scopes may use couple motion (e.g., the movement of one scope initiates movement of the second scope to maintain the coupled field-of-view of the two scopes). The coupled motion of the two scope may be maintained while the scopes exist in separate anatomic spaces (e.g., on either side of a tissue barrier, such as an organ wall) but are focused on the same tissue in between the scopes. The couple motion may be used when the two scopes are in the same space focused on the same field of view. In this case, the two scopes may cooperatively maintain a field of view that is larger than either scope is capable of capturing independently. A composite image may be created to display the larger field of view to a user. Synchronized motion may be used to maintain the spacing between the scopes to maintain the overall field of view.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example surgical operating room with endoscopic and laparoscopic devices.

FIG. 6 illustrates an example of a smart surgical scope adjusting imaging parameter(s) to maintain a coupled field of view with another imaging scope.

FIG. 7 illustrates an example of a smart surgical scope updating imaging parameter(s) to maintain a coupled field of view with another imaging scope.

FIG. 8 illustrates an example of emitting additional wavelengths through tissue to augment sensors in an endoscope.

FIG. 9 illustrates an example of using color distortion to indicate that an image is a composite image.

FIG. 10 illustrates an example method that may be performed by a surgical instrument.

### DETAILED DESCRIPTION

A more detailed understanding may be had from the following description, given by way of example in conjunction with the accompanying drawings.

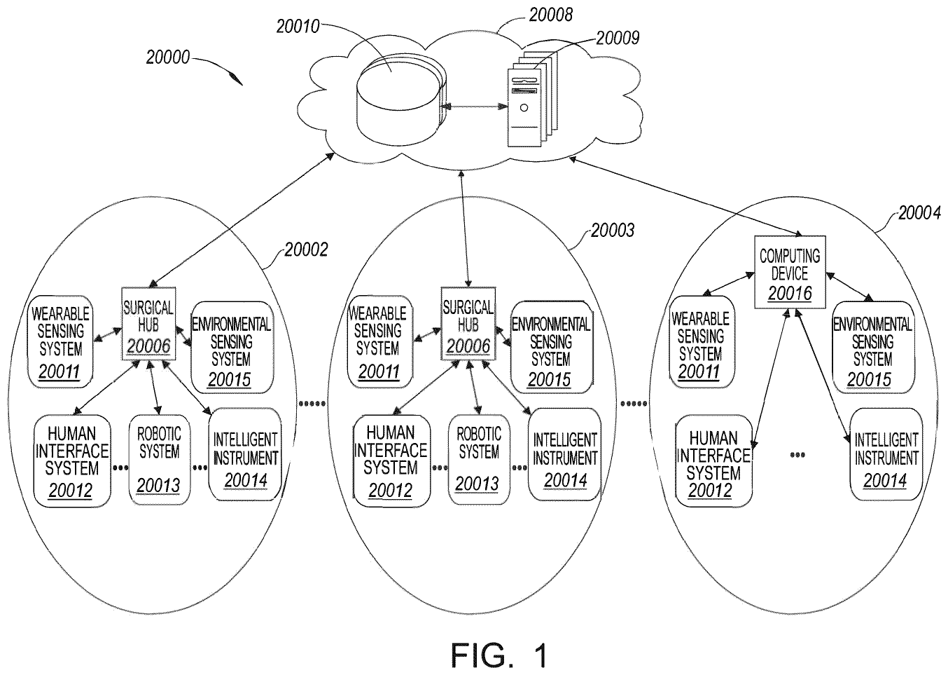

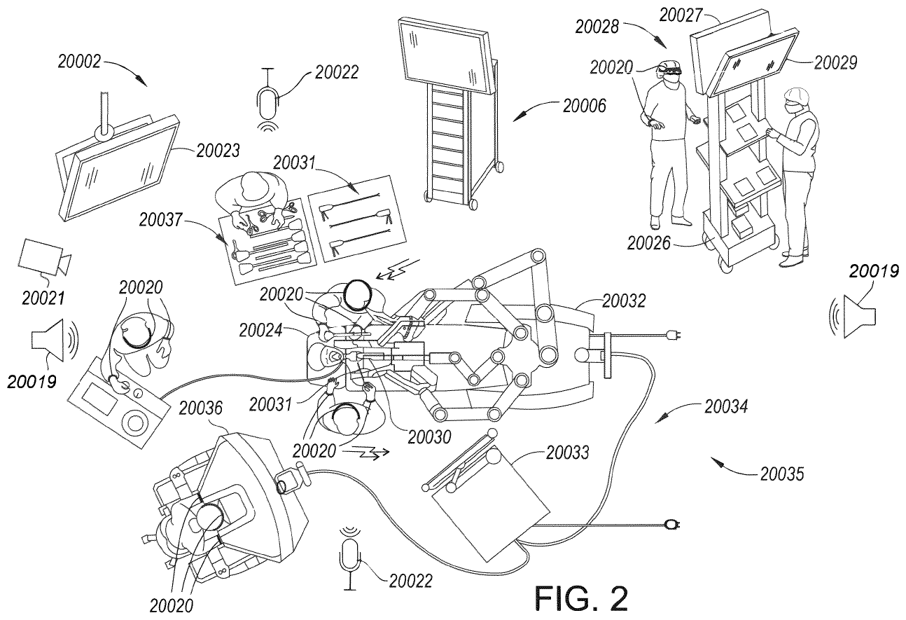

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

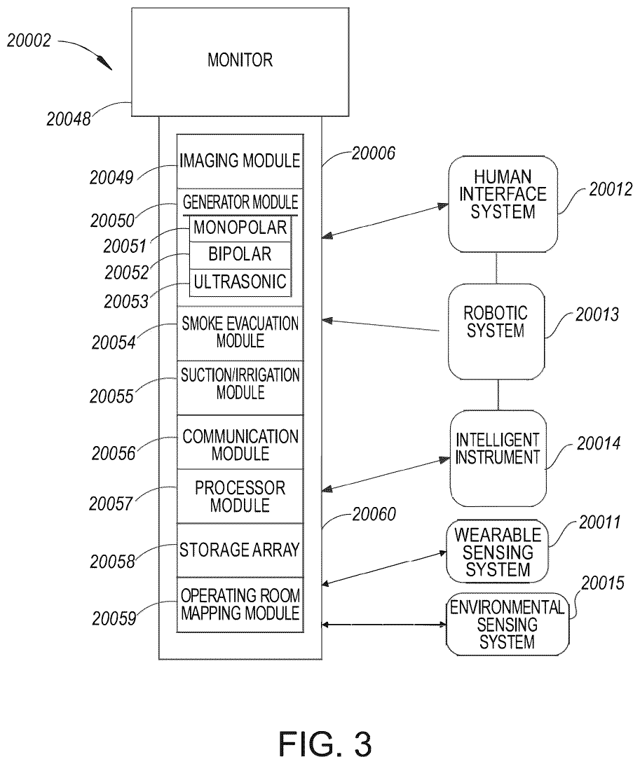

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

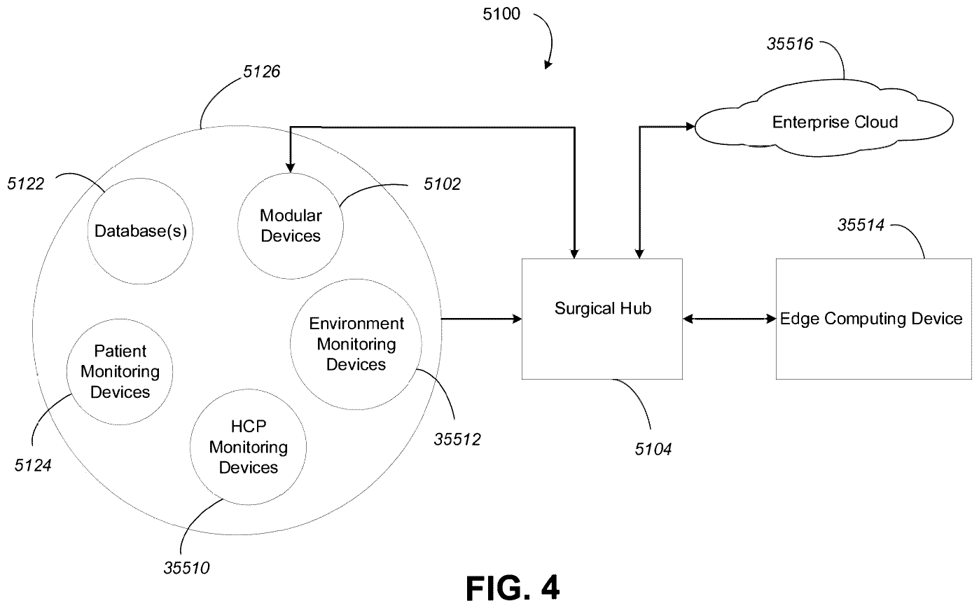

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

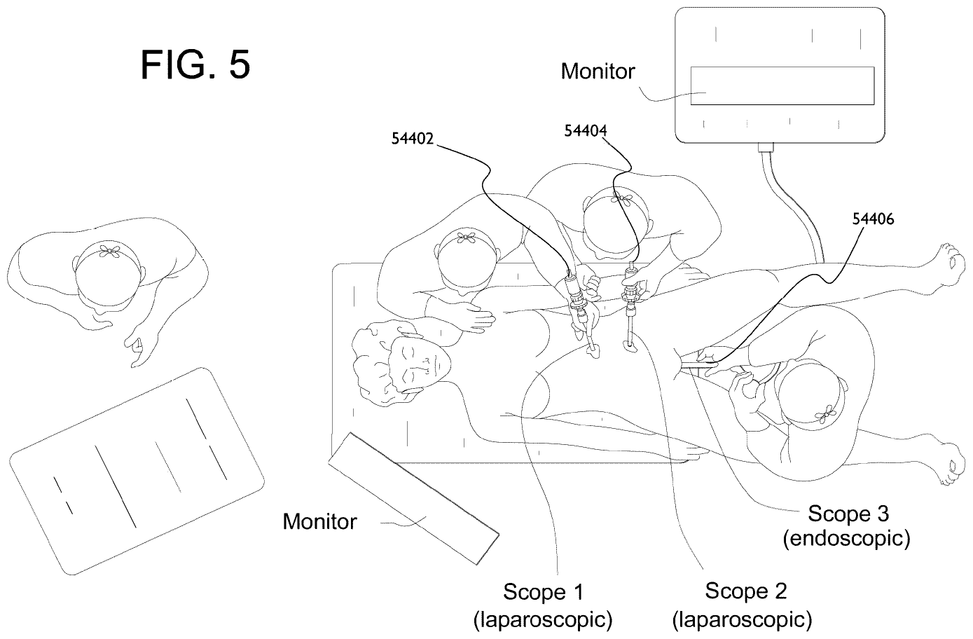

In operating rooms, multiple surgical imaging devices may operate in close proximity to one another. For example, FIG. 5 illustrates an operating room in which two laparoscopes 54402, 54404 and an endoscope 54406 are being used simultaneously. The imaging devices may all be from different manufacturers and may have different control systems. When devices, such as these, do not coordinate with each other they may limit the field of view of a user (e.g., surgeon). Such limited visibility may cause the user to miss important events during surgery, such as an unintended bleed. The coordination between and/or among imaging systems as disclosed herein may enhance the field of view of the user resulting, for example, in fewer missed surgical events and improving patient outcomes.

In an example, a first imaging device, such as an endoscope 54406, may include an imaging sensor and a processor. A second imaging device, such as any of laparoscopes 54402, 54404 may each include a respective imaging sensor and processor. The first and second imaging devices may be manual surgical instruments (e.g., as illustrated in FIG. 5). The first and second imaging devices may be robotic surgical instruments. The processor of the first imaging device may be configured to receive a video data stream captured by a second imaging device and determine, based on the video data stream, a coupled field of view between the first imaging device and the second imaging device.

A field of view may include a visual area that can be seen via an imaging device such as an endoscope 54406, and/or laparoscopes 54402, 54404. The field of view provided by the imaging devices may be dynamically adjustable. For example, the field of view may be adjusted by affecting changes in the device such as changes to the lens and/or optics, by changing the focal length of the optics, by affecting the zoom of the imaging device via physical optics and/or digitally, by affecting the effective sensor size (e.g., larger effective sensors generally have wider field of views), by affecting the position of the imaging device (e.g., physically moving the device may change the portion of the observable space; movements may include pan, tilt, shift, dolly, and the like), image stitching (e.g., multiple images may be stitched together to create a panoramic view to increase field of view), adjusting aperture size (e.g., to influence depth of field and perceived field of view), and the like.

In an example, imaging systems, for example a first and second imaging system, may coordination their respective operation. For example, the first and second imaging system may coordinate their respective operation regarding their respective fields of view. For example, synchronized imaging of two system may be used to maintain a common field-of-view or perspective for a plurality of systems (e.g., to transition objects from one field of view to another). Cooperative multiple scope synchronized motion may be used to maintain the relational field-of-views. The motion of multiple (e.g., two) independent imaging scopes may be coupled. The coupled motion may involve the movement of one scope initiating movement of a second scope to maintain the couple field-of-view of the two scopes.

In an example, the coordinate of fields of view may be implemented via the adjustment of an imaging parameter, as disclosed herein. For example, the processor of the first imaging device may determine, based on the video data stream, that the second imaging device has moved and may adjust an imaging parameter of the first imaging device to maintain the coupled field of view. The imaging parameter may include any parameter that represents a quantity suitable for altering a field of view of an imaging device, such as an electronically controlled field of view (e.g., digitally controlled resolution and/or windowing), a position of the first imaging device, a focal length of the first imaging device, a portion of a field of view associated with the first imaging device that is displayed to a user, and the like.

In the surgical environment, coordinated operation of two or more imaging systems regarding fields of view may be employed when the imagining systems are viewing within a common anatomical space and/or when the imaging systems are viewing within separate anatomical spaces. An anatomical space may include a cavity and/or compartment within the body. An anatomical space may include a space that contains organs, tissues, or other structures. The anatomical space may include a space accessible by a particular instrument. For example, in laparoscopic surgery, a small incision is made in the abdomen to insert a laparoscope which provides a view of the abdominal anatomical space (e.g., a laparoscopic anatomical space). For example, in endoscopic surgery, an endoscope may be inserted through natural orifice, such as the mouth or anus, and guided to the surgical site. Such spaces accessible in this way may include endoscopic anatomical space. In examples, the endoscope may be inserted through a small incision. In addition to surgical procedures, anatomical spaces may also be visualized using non-invasive medical imaging techniques such as X-rays and CT scans, for example.

In an example, with the two scopes in a common anatomical space (e.g., laparoscopes 54402, 54404 in a common laparoscopic space), coordination regarding field of view may include adjustments to a common observable area. In an example, the two scopes may cooperatively maintain a filed-of-view that is larger than either scope is capable of capturing alone. The composite image may be the entire displayed field-of-view (e.g., captured by both scopes). The synchronized motion may be used to maintain the spacing between the scopes (e.g., to maintain the overall field-of-view).

In an example, with two scopes in different anatomical spaces (e.g., either of laparoscopes 54402, 54404 in a laparoscopic space and endoscope 54406 in an endoscopic space) coordination regarding field of view may include adjustments to each scope's respective side of a common anatomical barrier between the two anatomical spaces. For example, visualization of the common anatomical barrier may be performed from differing sides (e.g., the endoscopic and laparoscopic sides) of a tissue wall (e.g., an organ wall). Coordinate of field of view may include adjusting the field of view to maintain viewing of opposite sides of the common anatomical barrier (e.g., maintaining respective views on both sides of the barrier in sync). For example, adjustments that affect the monitored position and/or orientation may be used to keep the two cameras focused on the same intermediate portion of a tissue wall. In an example, synchronization may be maintained through external imaging (e.g., a cone-beam computerized tomography or electromagnetic sensing of another camera).

Cone-beam computerized tomography (CBCT) may include a medical imaging technique that uses a cone-shaped X-ray beam to produce 3D images of the patient's body. CBCT is similar to traditional computed tomography (CT) but may use a lower radiation dose and may provide higher resolution images of the area of interest. CBCT can also be used to guide surgical procedures.

For example, CBCT may be used to determine the position of a surgical instrument during a procedure, such as the position of one or more scopes disclosed herein. Here, the tracking system of the surgical instrument may be configured to determine the position/orientation of each scope head (e.g., each camera's location) based on the relative position of the respective scope head as observed by the CBCT. The position information may be provided to a processor, which can be programmed or configured to determine, with the camera intrinsics, a complete coordinate model for each scope within a common geometry. Such a model may be used for registration and/or iterative tracking of the scopes.

Electromagnetic (EM) sensing may be used to determine the position/orientation of the one or more scopes disclosed herein. Each scope may include an EM sensor system. The sensor may include one or more conductive coils that are subjected to an externally generated electromagnetic field. When subjected to the externally generated electromagnetic field, each coil of the EM sensor system may produce an induced electrical signal having characteristics that depend on the position and orientation of the coil relative to the externally generated electromagnetic field. In an example, the EM sensor system may be configured and positioned to measure six degrees of freedom. e.g., three position coordinates X, Y, Z and three orientation angles indicating pitch, yaw, and roll of a base point or five degrees of freedom. e.g., three position coordinates X, Y, Z and two orientation angles indicating pitch and yaw of a base point. For example, the EM sensor system may include that disclosed in U.S. Pat. No. 6,380,732 (filed Aug. 11, 1999 and incorporated by reference herein). The position information may be provided to a processor, which can be programmed or configured to determine, with the camera intrinsics, a complete coordinate model for each scope within a common geometry. Such a model may be used for registration and/or iterative tracking of the scopes.

Field of view coordination may include the incorporation of certain visualizations to one and/or both of the video streams. Visualizations may include any operation to the visual information of the video stream of one imaging device based on information from the other imaging device. Here, coordination of the field of view includes the ability to operationally augment the field of view from one device with information from the other. For example, with imaging devices in different anatomical space, a visualization may include an operation to visually subtract, or make transparent, parts of the anatomical barrier (e.g., tissue wall) separating them. This may allow a user to section the common view and see depth, underlining structures, or instruments on the other side of the tissue wall.

In other examples, a multi-scope system may use visualization cooperative movement and/or an extended viewable display. Synchronized visualization may involve electronic and/or algorithmic field-of-view limiting and overlapping imaging to enable each camera to capture images to produce a composite image (e.g., by adapting the synchronized imaging arrays). The synchronization may employ misaligned (e.g., slightly misaligned) CMOS arrays (e.g., that image differing wavelengths of energy). Such an overlap mode of operation may enable the overlay of multi-spectral imaging over visible light imaging on a common display (e.g., with the different imaging originating from side-by-side CMOS arrays).

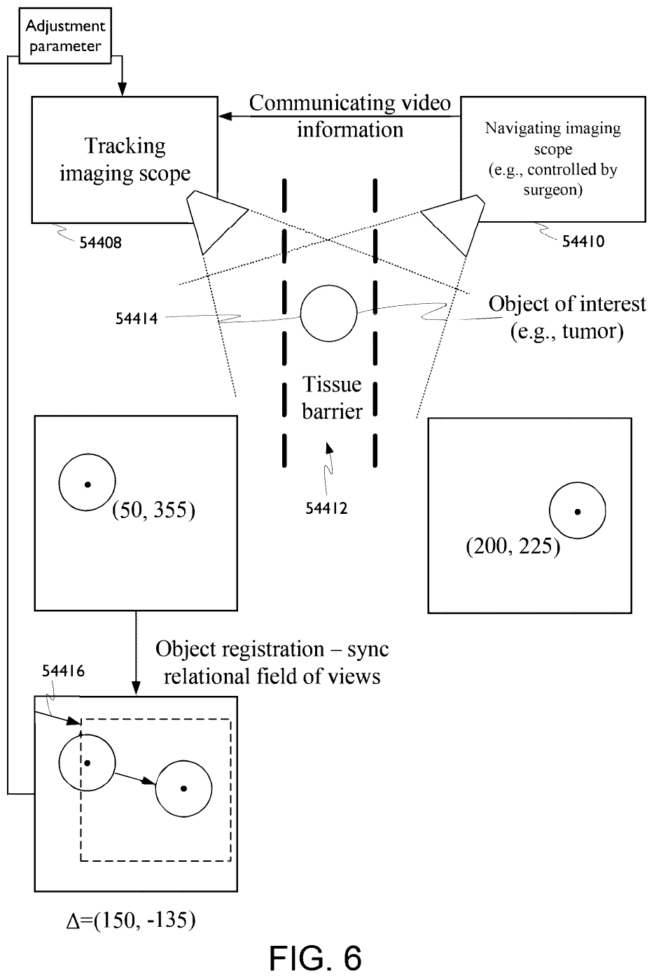

FIG. 6 illustrates an example of a first and second imaging devices with cooperative operation. Here, a tracking imaging scope 54408 may adjust an imaging parameter to maintain a coupled field of view with another imaging scope (e.g., a navigating imaging scope 54410). As shown, the two scopes may be on opposite sides of an anatomical barrier 54412 (such as a tissue barrier). A navigating imaging scope 54410 may be controlled by a surgeon. The navigating imaging scope 54410 may observe an object of interest 54414 (e.g., a tumor) and generate a corresponding video data stream.

The navigating imaging scope 55410 and the tracking imaging scope 54408 may exchange an initial handshake protocol to establish the corporative operation. The handshake protocol may include any data protocol suitable for device and capability discovery. For example, the handshake protocol may include a Universal Plug and Play UPnP protocol. Via UPnP, the navigating imaging scope 55410 and the tracking imaging scope 54408 may advertise their presence in the network and discover each other. The protocol may include sending responsive to discovery a description of available cooperative services available. The description may be an XML-based (Extensible Markup Language-based) description. The description may include identifying information about each device, such as manufacturer, model name, model number, serial number, and the like. The description may include capability-specific information including service type, service Id, Service description URL, control URL, eventing URL, and the like. The service description may include information about the device's capability to act as in a tracking and/or navigating capacity. The service description may include information regarding a destination for communicating video data stream information for purposes of cooperative field of view operation.

The navigating imaging scope 54410 may communicate the video data stream information to the tracking imaging scope 54408. The tracking imaging scope 54408 may receive the video data stream from the navigating imaging scope 54410.

Having video information from the navigating imaging scope 54410, the tracking imaging scope 54408 may establish a common coordinate system. To establish a common coordinate system, the system may use any computer vision, photogrammetry, and/or robotics-control techniques suitable for synthesizing information from multiple cameras. In an example, the system may use a computer vision algorithm to develop a common coordinate system. The tracking imaging scope 54410 may receive intrinsic parameter information from the navigating imaging scope 54408, for example, parameters such as focal length, principal point, lens distortion, and the like. The tracking imaging scope 54410 may receive extrinsic parameter information from the navigating imaging scope 54408, for example, parameters such as relative positions and/or orientations. In an example, one or more features or keypoints may be identified in both images. For example, the object of interest 54414, when in the field of view of both scopes, may be selected as such as feature.

The video data stream information may indicate coordinate points associated with the object of interest 54414. For example, as shown in FIG. 6, the navigating imaging scope 54410 may determine that the object of interest 54414 is centered at a point in the navigating imaging scope's field of view that corresponds to the coordinate point (200, 225). The tracking imaging scope 54408 may capture its own video information about the object of interest. As illustrated, for example, the tracking imaging scope 54408 may determine that the object of interest 54414 is centered at a point in the tracking imaging scope's field of view that corresponds to the coordinate point (50, 355). The difference in coordinate points between the scopes may be due to difference in one or more factors between the scopes, for example, angle of the camera, size of the field of view, location of a reference coordinate system origin, and/or the like. And, the difference in coordinate points may be accommodated with an adjustment to any of such factors.

For example, as shown in FIG. 6, the tracking imaging scope 54408 may perform object registration for the object of interest 54414. The tracking imaging scope 54408 may synchronize the relational field of views of the primary and secondary scopes, for example. The tracking imaging scope 54408 may synchronize the field of views by calculating the delta between the two coordinate points (e.g., in this example, Δ=(150, −135)). The tracking imaging scope 54408 may adjust one or more of its imaging parameters to establish and then maintain a coupled field of view with the navigating imaging scope. For example, the imaging parameter may include a physical location of the scope, an angle of view of the object of interest, a focal length of the scope, etc.

In an example, the tracking imaging scope 54408 may adjust an imaging parameter that includes an electronically controlled field of view, for example a digital window of its field of view, to establish a coupled field of view with the navigating imaging scope 54410. As illustrated in FIG. 6, the tracking imaging scope 54408 may determine the imaging parameter to be vector represented by the delta between the coordinate points identified in the object registration. The vector may be used to as a transform 54416 to be applied to the field of view of the tracking imaging scope 54408. The video image maybe translated and/or scaled in a manner proportional to the vector such that in the resulting field of view the objects of the object registration are aligned. The electronically controlled field of view may include digital windowing, CMOS segment activation/deactivation, optical and/or digital zoom, aperture control, focal length, and the like.

In an example, the tracking imaging scope 54408 may adjust an imaging parameter, for example the position of the camera of the tracking imaging scope 54408, to establish a coupled field of view with the navigating imaging scope 54410. Again, the tracking imaging scope 54408 may determine the imaging parameter to be vector represented by the delta between the coordinate points identified in the object registration. The vector may be used to as a control input to be applied to a surgical robot control of the tracking imaging scope 54408. The change in position of the camera may be proportional to the vector such that in the resulting field of view the objects of the object registration are aligned.

In an example, the tracking imaging scope 54408 may adjust more than one imaging parameter, for example the position of the camera of the tracking imaging scope 54408 and a digital window of its field of view, to establish a coupled field of view with the navigating imaging scope 54410. For example, the adjustment may apportion the vector represented by the delta between the coordinate points identified in the object registration to the more than one imaging parameters. The proportional adjustment of each may result in a field of view in which the objects of the object registration are aligned.

In an example, adjustment of the imaging parameter(s) may be done in unit steps, iteratively, gradient-based, or the like. In an example, the adjustment of the imaging parameter(s) may incorporate modeled aspects of the tracking imaging scope's performance, such a project camera model. Here, a project camera model with camera intrinsics and inverse pose may be solved to determine a world line corresponding to the image point corresponding to the tracking imaging scope's object identified in the object registration. Then, a new set of intrinsics and/or inverse pose may be solved for using the same world line and the target image point represented by the delta between the objects identified in the object registration.

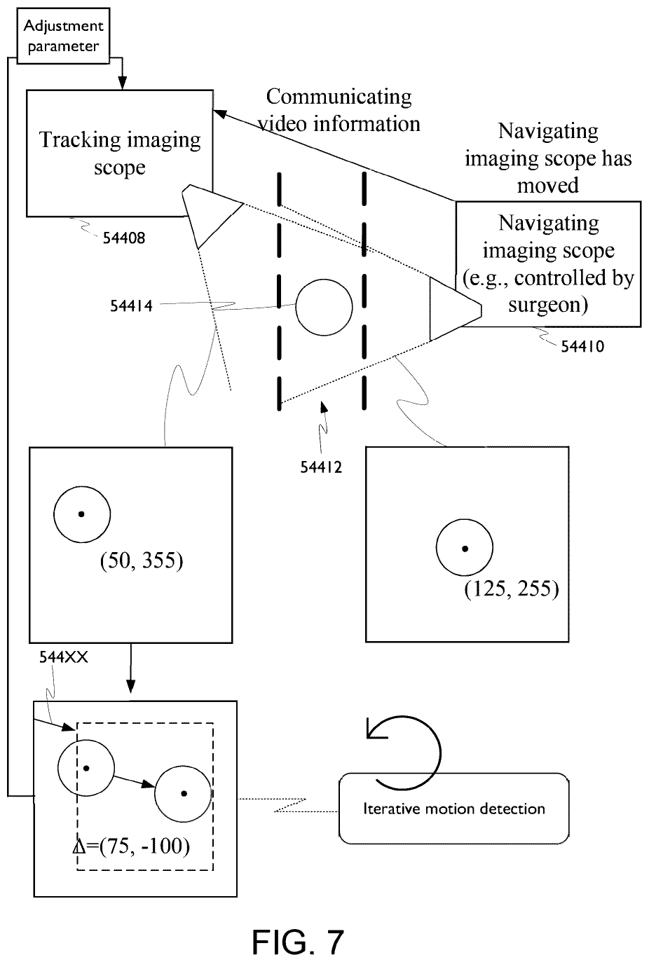

FIG. 7 illustrates an example of a smart surgical scope updating imaging parameter(s) to maintain a coupled field of view with another imaging scope. As shown in FIG. 7, the navigating imaging scope 54410 may have moved (e.g., compared to its location in FIG. 6). The navigating imaging scope 54410 may continue to communicate its video data stream information to the tracking imaging scope 54408. The tracking imaging scope 54408 may detect the navigating imaging scope's movement based on the video data stream information. For example, the video feed information may indicate a different coordinate point associated with the object of interest than previously reported. For example, the tracking imaging scope 54408 may use video-based motion estimation of the video data stream information to detect the navigating imaging scope's movement. The video-based motion estimation may include approaches such as block matching algorithms, optical flow, phase correlation, gradient-based methods, Kalman filtering, and the like.

The tracking imaging scope 54408 may compare subsequent video images to determine a difference between the coordinate points of the two scopes (e.g., in this examples, Δ=(75, −100)) after the movement of the navigating imaging scope 54410. The tracking imaging scope 54408 may adjust one or more of its imaging parameter(s), as described herein. Adjusting the imaging parameter(s) will change the coordinate point that the secondary imaging device associates with the object of interest. The tracking imaging scope may then iteratively compare the coordinate points of the two scopes and adjust imaging parameter(s) as needed until the fields of view are synchronized. The tracking imaging scope 54408 may determine the coupled field of view has been maintained on the condition field of the view of the tracking imaging scope 54408 and the video data stream are aligned.

In an example, the navigating imaging scope 54410 may communicate information indicative of its movement to the tracking imaging scope 54408. For example, a tracking imaging scope 54408 driven by a surgical robot control may provide the control inputs given by the surgeon to the surgical robot to the tracking imaging scope 54408 via a data channel (e.g., embedded in the video data stream and/or a channel separate from the video data stream). The tracking imaging scope 54408 may use the control inputs when determining the motion seen in the video data stream. In an example, other positioning data such as surgical x-ray, cone CT, EM sensing, the like, may be employed to determine motion.

In an example, other aspects of scope operation may be coordinated to enhance performance and/or usability. Operational aspects such as lighting intensity, wavelength, display features, and the like may be coordinated via communication between the scopes. For example, the motion of each scope and/or the direction of the motion from the light source from each individual scope may be used in cooperation (e.g., to enhance the view of each scope). Synchronizing the motion of the scopes on the surgical site and the light source of a scope may be used to enhance the view from the other scope. A laparoscopic scope may limit the luminescence or the amount of light the scope generates. The light source may be directed from behind the camera of the scope. During a procedure utilizing two scopes, the light sources of each camera may be synchronized to benefit the other. The light source of the scope may obscure the lighting back to the field of view (e.g., based on the surgical location and/or other instruments). In this case, the first scope may turn off its light source. The second scope may be used to direct its light source to optimize the view. Each scope may have unique wavelengths and/or color (e.g., to further optimize the feedback to the user).

Regarding display aspects, in an example, camera views may be altered (e.g., by establishing the same coordinate system for the two scopes, as described herein). The coordinate system synchronization may allow a surgeon to easily toggle back and forth between the two scopes, while still recognizing what is shown in both views. A user may be able to swap between primary and secondary live feeds. For example, there may be a solid part or organ within the surgeon's field of view. The system may allow the surgeon to switch to another field of view associated with a different scope.

In addition, scope coordination may enhance surgical operation through enhanced calibration. For example, calibration may be enabled via multi-system registration of common object. And a redundant imaging system may be used to view a common object between two systems (e.g., one of which is being affected by navigation distortion) to aid in the compensation for calibration. For example, the laparoscopic and endoscopic imaging systems may be coupled together for cooperative motion. And the cooperative motion may be used to enable one of the two visualization systems that is highly impact by distortion (e.g., an electromagnetic navigation system of the robotic flexible endoscopic system) to track common landmarks or easily identified structures (e.g., to help correct for the distortions in its navigation system without the need for frequent radio transmissive recalibration). The relational field-of-views position may indicate a global position of the position of a tumor. The system may use the global position of the tumor to augment the view of the tumor into the laparoscopic field of view. As the colon (or hollow organ, e.g., stomach, bladder, etc.) moves, the image recognition from the laparoscopic side (e.g., through TOF, structured light, or optical shape sensing) and proximity of the endoscope may be used to dynamically move the augmented tumor view.

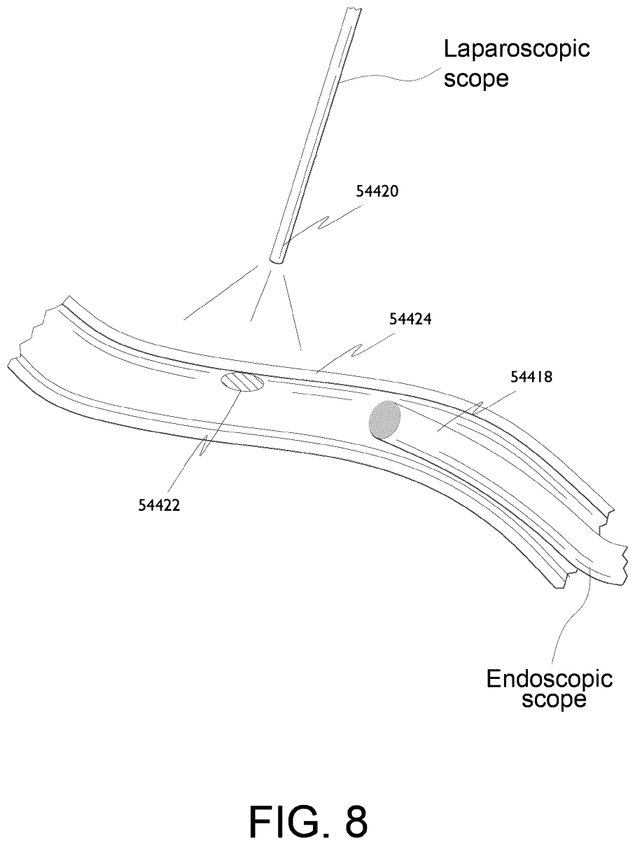

FIG. 8 illustrates an example of emitting additional wavelengths from one scope through tissue to augment sensors in another scope. As illustrated, the laparoscopic scope 54420 may shine a light toward an object of interest 54422 on the opposite side of a tissue barrier 54424. The endoscopic scope 54418 may have a direct view of the object of interest 54422. The endoscopic scope 54418 may be able to detect the wavelengths of the light from the laparoscopic scope 54420. The wavelengths, as shown through the object of interest 54422, may inform the surgeon of one or more properties of the object of interest.

In an example, an illumination-providing scope or light source may be moved in advance of the imaging scope (e.g., to compliment the illumination of the imaging scope). In this predictive operating mode, the light source may be moved first (e.g., to remove shadows and improve visualization of recessed areas that the light from the primary scope has not yet illuminated). Using FIG. 8 as an example, the endoscopic scope 54418 may be placed first and may illuminate the location of the object of interest 54422. The laparoscopic scope 54420 may use the light from the endoscopic scope 54418 to locate the object of interest 54422 (e.g., even though it does not have a direct view of the object).

In an example, a visualization may be generated in the video output of one of the scopes based on information from the other scope. For example, the visualization may include information from a first imaging device that when incorporated into the video output of the second imaging device makes a portion of the viewed anatomical barrier appear transparent. To illustrate, video of the object of interest 54422 taken from the endoscopic scope 54418 may be overlaid the video from the laparoscopic scope 54420 to reveal an image of the object of interest 54422 in the laparoscopic view. As modified, a portion of the tissue barrier 54424 in the video may appear to be transparent. Such video operation may include a selective subtractive image modification operation.

Examples of procedures in which the techniques described with respect to FIG. 8 may be implemented are described in greater detail in U.S. Patent Application Publication No. US 2023-0095002 A1 (U.S. patent application Ser. No. 17/493,913), titled SURGICAL METHODS USING FIDUCIAL IDENTIFICATION AND TRACKING, filed Oct. 5, 2021, the disclosure of which is herein incorporated by reference in its entirety.

In an example, subtractive image modification may be used to fill in the shapes and colors of the part of the image that was occluded. Subtractive image modification may be used to enable user visibility of occluded areas. Subtractive image modification may be used to enable visualization of occluded areas.

Cooperative operation among imaging devices may enable the presentation of the image(s) modified with color. For example, an area made transparent (e.g., by subtractive image modification) may be shaded with a color. The area that is visible due to the transparency may be shaded with color. The image may be composed (or decomposed) into the relative areas and related colors. For example, a primary image may be blue and a secondary image may be red. The primary and secondary images may be composed into the blind spot/occluded image (e.g., which may be purple). The color may be distorted (e.g., using software) on each camera to be shaded with a different tint (e.g., red or blue to make it more obvious when a composite image is seen).

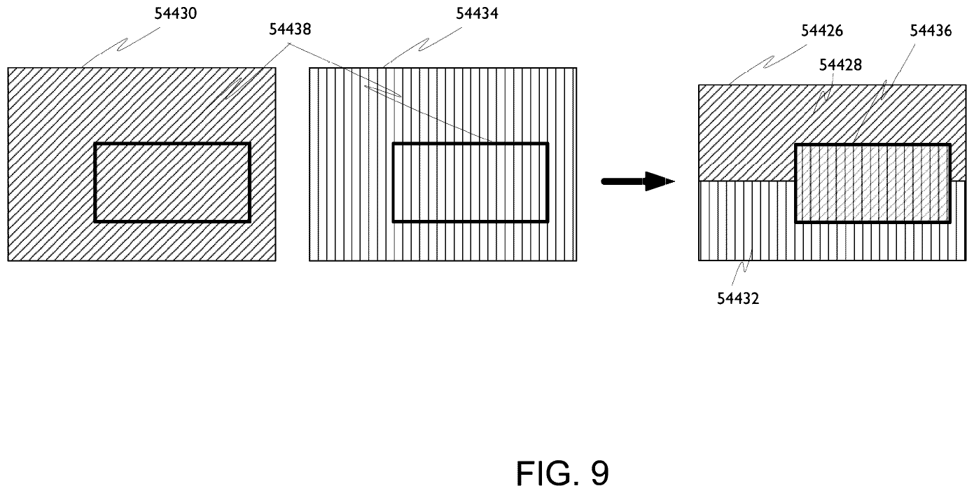

FIG. 9 illustrates an example of using color distortion to indicate that an image is a composite image. For example a modified output video stream 54426 may have three portions. The first portion 54428 may represent corresponding video from video stream 54430 from a first imagine device. The second portion 54432 may represent corresponding video from video stream 54434 from a second imagine device. And the third portion 54436 may represent an overlay of a corresponding portion 54438 from both the video stream 54430 from the first imaging device and the video stream 54434 from the second imagine device. The composite image may use one or more such portions 54428, 54432, 54436 in any combination and/or orientation. The source identity of the portions 54428, 54432, 54436 may be indicated to the user by color. For example, the first portion 54428 may be a first color that corresponds to the video stream 54430 from the first imagine device. The second portion 54432 may be a second color that corresponds to the video stream 54434 from the second imagine device. And the third portion 54436, as an overlay, may be a third color, for example, a color that represents a proportional mix of the first and second colors.

In an example, a selective view of certain segments may be used. The ability to make the anatomy transparent may be enabled/disabled (e.g., by a user). The system may allow the surgeon to draw shapes around the areas they would like to be made transparent (e.g., using a tool such as a tablet).

Synchronization of multi-sourced imaging data (e.g., currently active vision systems or prior scans) may be used to generate or create a virtually-rendered field of view. This enables a field of view larger than that possible using a single vision system or overlapping. Vision systems may coordinate and alternate performing scans of the surrounding area (e.g., to determine whether to update the virtually rendered field of view). For example, a first vision system, vision system A, may be the currently active vision system. A second vision system, vision system B may run a scan of surrounding area. Vision system B may update the combined field of view (e.g., if necessary). If vision system B's area of view is currently active, vision system A may perform the sweeping scan to check for any field of view updates.

In an example, data input from multiple sources may be used to enable multi-directional modality projection. Multi-directional projection of energy modalities (e.g., light spectrum, gamma, x-ray, microwave) may be used. Each source may include a respective imagine sensor that senses light of a corresponding band. So a video data stream captured from one scope may then represent light from a first band, and a video stream captured by another scope may represent light of a second band that is different from the first band. The light from differing scopes and/or projectors may be received. Penetrating or backlit cooperative scope wavelength motion and detection system may introduce wavelengths, intensities, and/or combined wavelengths (e.g., for which the first scope may not have the bandwidth or capabilities). In the case of high intensity coherent light sources, the fiber optics of the shaft may conduct the light from a source outside of the body. As the size of the shafts gets smaller, the fiber bundles may get smaller. This may cause the fiber bundles to be overloaded with the magnitude of the energy being conducted (e.g., often melting or degrading the fiber optics). These externally-projected lights may be introduced with a larger system (e.g., from the lap side with a 12 mm shaft), for example, as opposed to the sending system (e.g., a 2 mm CMOS arrays on the flexible scope side). This may provide illumination to sub-surface structures (e.g., because the light is projected through the tissue rather than being projected onto the tissue). The larger system may allow the projection source to be stronger (e.g., orders of magnitude stronger) than the 2 mm fiber optics could provide. The projection source may be in addition to the local light imaging of the endoscopic scope (e.g., thereby minimizing the extra-spectral loss of the magnitude of the visible light spectrum). The projection source light may include wavelengths and/energy sources that are not compatible with the flexible scope system (e.g., wavelengths exciting a reagent, for example, ICG or tumor tagging fluorophores with wavelengths that cause excitation of the natural molecular nature of the tissue, radiation, microwave, etc.).

The movement of one or more visualization system(s) and/or smart device(s) may be synchronized so that the visualization system(s) are paired to the device. The movement of a device may initiate movement of one or more cameras (e.g., to maintain a common field of view). Vision systems may track a smart device to maintain field of view (e.g., using a sub-spectral or infrared tag). Vision systems may not (e.g., may not need to) keep a device centered in its field of view (e.g., if AE or critical area of tissue gains precedence, in which case the AE or critical area of tissue may be the center of the field of view).

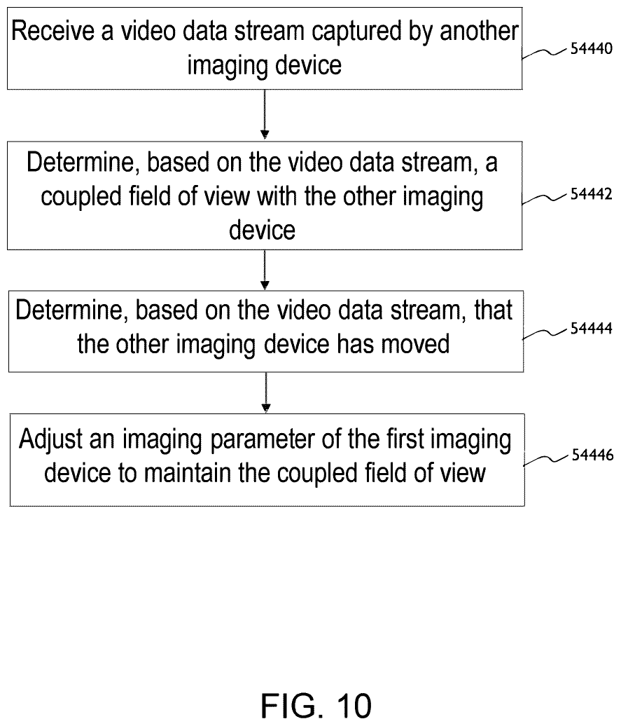

FIG. 10 illustrates an example method that may be performed by a surgical instrument. At 54440, a video data stream captured by a second imaging device may be received by a first imaging device. For example, the first imaging device may be located on a first side of an anatomical barrier, and the second imaging device may be located on a second side of the anatomical barrier. The first side and the second side are opposing sides of the anatomical barrier. For example, the anatomical barrier may include tissue that defines a first anatomical space within which is the first imaging device and that defines a second anatomical space within which is the second imaging device. For example, an endoscopic view may be generated by a video stream captured by the first imaging device. The video stream captured by the second imaging device may represent a laparoscopic view. The first imaging device may include a first imaging sensor that senses light of a first band. The second imaging device may include a second imaging sensor that senses light of a second band. The first band may be different from the second band.

The reception of the video data stream may be responsive to a handshake protocol. For example, the first imaging device may communicate a handshake protocol with the second imaging device to establish cooperative operation.

At 54442, a coupled field of view may be determined, based on the video data stream. In an example, the coupled field of view may be determined by performing an object registration on an object and synchronizing a first field of view associated with the first imaging device with a second field of view associated with the second imaging device based on the object registration. Here, the object may be visible in the first field of view and in a second field of view. In an example, the coupled field of view may be determined by receiving relative field of view positioning information from cone-beam computerized tomography imaging that comprises a view of the first imaging device and the second imaging device. In an example, the coupled field of view may be determined by receive relative field of view positioning information from electromagnetic sensing of the second imaging device.

In an example, a visualization associated with the anatomical barrier in the video data stream may be generated based on information from an imaging sensor of the first imaging device. For example, the visualization may include information from the imaging sensor of the first imaging device that makes a portion of the anatomical barrier appear transparent.

At 54444, it may be determined, based on the video data stream, that the second imaging device has moved. And at 54446, an imaging parameter of the first imaging device may be adjusted to maintain the coupled field of view. In an example, the imaging parameter may include an electronically controlled field of view. In an example, the imaging parameter may include any of a position of the first imaging device, a focal length of the first imaging device, or a portion of a field of view associated with the first imaging device that is displayed to a user. The imaging parameter may be adjusted to maintain the coupled field of view by iteratively comparing a first field of view associated with the first imaging device to the video data stream and adjusting the imaging parameter based on the comparison. It may be determined that that the coupled field of view is maintained based on the condition that the first field of view and the video data stream are aligned with respect to a registered object.

What is claimed:

1. A first imaging device comprising a processor, wherein the processor is configured to:
receive a video data stream captured by a second imaging device, wherein the first imaging device is located on a first side of an anatomical barrier, wherein the second imaging device is located on a second side of the anatomical barrier, and wherein the first side and the second side are opposing sides of the anatomical barrier;
determine, based on the video data stream, a coupled field of view between the first imaging device and the second imaging device;
determine, based on the video data stream, that the second imaging device has moved; and
adjust an imaging parameter of the first imaging device to maintain the coupled field of view.

2. The first imaging device of claim 1, wherein the anatomical barrier comprises tissue that defines a first anatomical space within which is the first imaging device and a second anatomical space within which is the second imaging device.

3. The first imaging device of claim 1, wherein the processor is further configured to generate an endoscopic view and wherein the video stream captured by the second imaging device represents a laparoscopic view.

4. The first imaging device of claim 1, wherein the processor is further configured to generate a visualization associated with the anatomical barrier in the video data stream based on information from an imaging sensor of the first imaging device.

5. The first imaging device of claim 4, wherein the visualization comprises information from the imaging sensor of the first imaging device that makes a portion of the anatomical barrier appear transparent.

6. The first imaging device of claim 1, wherein the processor being configured to determine a coupled field of view between the first imaging device and the second imaging device comprises the processor being configured to:
perform object registration on an object, wherein the object is visible in a first field of view associated with the first imaging device and in a second field of view associated with the second imaging device; and
synchronize the first field of view with the second field of view based on the object registration.

7. The first imaging device of claim 1, wherein the processor being configured to determine a coupled field of view between the first imaging device and the second imaging device comprises the processor being configured to:
receive relative field of view positioning information from cone-beam computerized tomography imaging that comprises a view of the first imaging device and the second imaging device.

8. The first imaging device of claim 1, wherein the processor being configured to determine a coupled field of view between the first imaging device and the second imaging device comprises the processor being configured to:
receive relative field of view positioning information from electromagnetic sensing of the second imaging device.

9. The first imaging device of claim 1, wherein the imaging parameter comprises an electronically controlled field of view.

10. The first imaging device of claim 1, wherein the imaging parameter comprises any of a position of the first imaging device, a focal length of the first imaging device, or a portion of a field of view associated with the first imaging device that is displayed to a user.

11. The first imaging device of claim 1, further comprising an imaging sensor that senses light of a first band, wherein the video data stream captured by the second imaging device represents light of a second band that is different from the first band.

12. The first imaging device of claim 1, wherein the processor being configured to adjust the imaging parameter of the first imaging device to maintain the coupled field of view comprises the processor being configured to:
iteratively compare a first field of view associated with the first imaging device to the video data stream, and adjust the imaging parameter based on the comparison; and
on a condition that the first field of view and the video data stream are aligned with respect to a registered object, determine that the coupled field of view is maintained.

13. The first imaging device of claim 1, wherein the processor is further configured to communicate a handshake protocol with the second imaging device to establish cooperative operation.

14. A method comprising:
receiving, by a first imaging device, a video data stream captured by a second imaging device, wherein the first imaging device is located on a first side of an anatomical barrier, wherein the second imaging device is located on a second side of the anatomical barrier, and wherein the first side and the second side are opposing sides of the anatomical barrier;
determining based on the video data stream, a coupled field of view between the first imaging device and the second imaging device;
determining, based on the video data stream, that the second imaging device has moved; and
adjusting an imaging parameter of the first imaging device to maintain the coupled field of view.

15. The method of claim 14, wherein the anatomical barrier comprises tissue that defines a first anatomical space within which is the first imaging device and a second anatomical space within which is the second imaging device.

16. The method of claim 14, wherein the method further comprises generating an endoscopic view and wherein the video stream captured by the second imaging device represents a laparoscopic view.

17. The method of claim 14, wherein the method further comprises generating a visualization associated with the anatomical barrier in the video data stream based on information from an imaging sensor of the first imaging device.

18. The method of claim 17, wherein the visualization comprises information from the imaging sensor of the first imaging device that makes a portion of the anatomical barrier appear transparent.

19. The method of claim 14, wherein the imaging parameter comprises an electronically controlled field of view.

20. The method of claim 14, wherein the imaging parameter comprises any of a position of the first imaging device, a focal length of the first imaging device, or a portion of a field of view associated with the first imaging device that is displayed to a user.
